(19) United States
(12) Patent Application Publication
(10) Pub. No.: US 2025/0049515 A1
(43) Pub. Date: Feb. 13, 2025

(54) SURGICAL ROBOT SYSTEM AND CONTROL METHOD

(71) Applicant: B. Braun New Ventures GmbH, Freiburg im Breisgau (DE)

(72) Inventor: Amir Sarvestani, Freiburg (DE)

(21) Appl. No.: 18/718,863

(22) PCT Filed: Dec. 12, 2022
(86) PCT No.: PCT/EP2022/085447

§ 371 Date: Jun. 12, 2024

(30) Foreign Application Priority Data
Dec. 14, 2021 (DE) 10 2021 133 060.2

(51) Int. Cl. A61B 34/30; A61B 1/00; A61B 1/05; A61B 1/317; A61B 34/20; A61B 90/14

(52) U.S. Cl. CPC A61B 34/30; A61B 1/00149; A61B 1/317; A61B 90/14; A61B 1/05; A61B 2034/2055; A61B 2034/2065; A61B 2034/302

(57) ABSTRACT

A surgical robot system, a control method and a computer-readable storage medium can be used with or for a surgical procedure on a patient. The system can include at least one patient fixation unit adapted to be rigidly and directly attached to the patient to rigidly fix at least one body part of the patient with an intervention region relative to the patient fixation unit. At least one controllable surgical robot has a robot base directly connected to the patient fixation unit, and a movable robot arm connected to the robot base. An end effector, in particular a surgical instrument, can be connected to, in particular mounted on, the robot arm, in particular to a terminal side of the robot arm.

### CROSS-REFERENCE TO RELATED APPLICATIONS

This application is the United States national stage entry of International Application No. PCT/EP2022/085447, filed on Dec. 12, 2022, and claims priority to German Application No. 10 2021 133 060.2, filed on Dec. 14, 2021. The contents of International Application No. PCT/EP2022/085447 and German Application No. 10 2021 133 060.2 are incorporated by reference herein in their entireties.

### FIELD

The present disclosure relates to a surgical robot system for a surgical intervention on a patient, wherein a controlled end effector participates in the intervention. Furthermore, the present disclosure relates to a control method/assistance method and a computer-readable storage medium.

### BACKGROUND

Robot-assisted surgical systems for assisting surgical interventions are widely used in particular in the field of laparoscopic surgery. Surgical robots or robot systems are also being used more and more in neurosurgery, spinal surgery and ENT surgery. However, one disadvantage of current robot systems is that they are almost exclusively ground-mounted robot systems, in exceptional cases also ceiling-mounted robot systems, whose configuration has a negative effect on the actuation of an end effector used. In this context, ground-mounted or ceiling-mounted means that the local connection of the robot to the system fixed it the room and thus to the patient with the corresponding intervention region is made via the floor or ceiling of the operating room. In the case of ground-mounted robot systems, for example, mobile units are known which can be placed in the operating theater at various positions relative to the operating table on which the patient is lying in order to realize various relative arrangements to the latter.

However, a decisive disadvantage of these robot systems is that the accuracy is severely limited due to unavoidable movements between the robot base of the robot and the patient and often does not meet the minimum requirements for precision. The further/longer the geometric, direct connection (with corresponding force flow) between the patient on the one hand and the robot end effector on the other hand, the higher this inaccuracy becomes due to, for example, elastic deflections, a lack of precision with regard to a calculated position and other disadvantageous influencing variables. Although solutions have been developed for a robot system or a robot that can be fastened to an operating table, these solutions are only suitable for an intervention to a limited extent, since at least the inaccuracy remains due to patient movement and insufficient fastening to the operating table.

In the prior art, there are therefore also ground-mounted robot systems that are first positioned and then establish an additional rigid reference connection between the robot base and a head holder of the patient in order to further increase accuracy. However, such ground-mounted robot systems are complex, expensive to produce and also take up a large area in the operating theater. Furthermore, a long path of force flow remains. In particular, these robots only guide a trajectory, for example to place a biopsy, while the surgeon continues to move the instrument manually. Robot systems are also known from the prior art that can be used to move instruments remotely in neurosurgery, but these systems are unfavorably mounted on the floor, with an associated loss of precision, and it is also not possible to track the position of the instrument used in the intervention. Furthermore, robot systems are known that track the position of an instrument with the aid of a (standard) navigation system that uses, for example, optical tracking via a rigid body or electromagnetic tracking (EM tracking). However, attaching a rigid body to the instrument itself decreases its ergonomics and increases the volume of work. Furthermore, an EM system has the disadvantage of distortions caused by devices that emit electromagnetic radiation of a similar wavelength. This EM radiation can, for example, be generated by the robot itself, which ultimately has a negative impact and impairs tracking accuracy.

### SUMMARY

It is therefore the object of the present disclosure to avoid or at least reduce the problems of the prior art and in particular to provide a surgical robot system which provides a particularly high precision of a controlled end effector during an intervention and which is safe and easy to operate. An improvement or shortening of an operation time is also to be achieved and the user, in particular the surgeon, is to be provided with a high level of operating comfort. In addition, a partial object can be seen in making the configuration simple and modular and enabling cost-effective production. Another partial object is to be able to place and align a robot even better and more flexibly in relation to an intervention region in order to be able to perform the intervention even better and more easily.

The objects of the present disclosure are solved with respect to a generic surgical robot system according to the present disclosure, with respect to a generic (robot) control method according to the present disclosure, and with respect to a computer-readable storage medium according to the present disclosure.

A basic idea of the present disclosure can be seen in that a geometric frame structure/fixing structure (patient fixation unit) is immediately and directly connected to the body part of the patient on which the surgical intervention takes place and is statically fixed, in particular rigidly relative to a portion of the human skeleton. In this way, when the patient is moved, a relative transformation between a local coordinate system (KOS) of the patient or the intervention region on the patient on the one hand and a local KOS of the patient fixation structure on the other hand is not or only minimally changed and remains static even when the fixed body portion of the patient is moved. The surgical robot in turn is fastened or coupled directly to this geometric fixing structure via its robot base, so that a relative transformation between the patient with the intervention region and the robot base is also kept constant and does not change if the patient is moved on the operating table, for example. This special configuration significantly increases the precision of the intervention. Therefore, no floor-supported robot, no ceiling-supported robot and no table-supported robot is used, but rather a patient-supported robot, so to speak, in order to establish a direct connection with the patient without having to reach over the operating table itself. The reference for the local coordinate system of the robot or the robot base is therefore not the floor or the table, but (via the patient-fixing system) the patient or the body portion of the patient to be fixed.

In other words, a surgical robot system for a surgical intervention on a patient is disclosed, comprising: at least one patient fixation unit which is adapted to be rigidly and directly connected/attached to the patient, in particular to a head of a patient, in order to rigidly fix at least a body portion of the patient with an intervention region relative to the patient fixation unit; at least one surgical robot having a robot base, wherein the robot base is directly and immediately connected, in particular attached or coupled, to the patient fixation unit, as well as having a movable robot arm connected to the robot base, and having an end effector, in particular a surgical instrument, connected to the robot arm, in particular to a terminal side of the robot arm, and is in particular mounted thereto. This robot system allows particularly precise and intuitive control of an end effector. For example, an instrument or an image device such as a camera can be used as the end effector in the surgical robot system.

In this context, a direct connection means that the immediate (serial) geometric connection of the at least one surgical robot is made directly to the patient fixation unit, and this connection is independent of a connection to an object in the operating room, such as an operating table or a floor. Although the patient fixation unit may also be connected to the operating table in order to achieve further stabilization, the (serial, shortest) connection between the patient and the end effector is not via the operating table or a floor-supported system, but directly and solely via the patient fixation unit. In this way, a direct path between the end effector and the patient can be kept particularly short and, as a result, precision and control can be improved.

In yet other words, the present disclosure relates to a miniaturized surgical robot system for minimally invasive surgery in which at least one robot is directly coupled/connected/attached to a patient-fixing system with a patient fixation unit, wherein the one robot with a robot arm or also several robots with respective robot arms is/are directly connected to the patient fixation unit itself. The robot arms each guide the end effectors, in particular surgical instruments and/or visualization devices. In yet other words, a surgical robot system for minimally invasive surgery is proposed in which at least one robot with a robot arm, in particular two robots, is attached directly to a patient fixation unit and guides the at least one end effector, in particular the surgical instruments and/or visualization device, via the robot arm.

Due to the special configuration, the robots with the robot arms can be designed to be particularly space-saving and small in size and still deliver outstanding precision during use. Particularly good articulation can also be ensured, since the attachment point of the robot base is particularly close to the intervention region.

The advantages of the present disclosure therefore lie in the improvement of precision, a reduction in operating time due to simpler and faster interventions and an increase in operating comfort for a surgeon in minimal surgery, in particular in the fields of neurosurgery, spinal surgery and ENT surgery. The instruments do not have to be guided manually, as the movements are carried out by a robot, thus opening up a telemanipulation approach. The disclosure presented here is thus the first surgical robot system that enables robotic teleoperation in neurosurgery, spinal surgery and ENT surgery with miniaturized robotic arms working through a very small port.

Advantageous embodiments are explained in particular below.

According to an embodiment, the surgical robot system may further comprise: at least one optical image unit, which is adapted to create an optical image of the intervention region, in particular with an intracorporeal tissue of the patient, and an end-effector tip, in particular an instrument tip, in the field of vision of the image unit and to provide it in a computer-readable manner/digitally; a data provision unit, in particular a memory unit, which is adapted to provide digital 3D image data, in particular preoperative 3D image data such as a computed tomography or a magnetic resonance imaging, of the patient; a tracking system, in particular a surgical navigation system, which is provided and adapted to detect and track in space at least the optical image unit and at least directly or indirectly the body portion of the patient, which is fixed relative to the patient fixation unit; and a control unit, which is provided and adapted to determine a position, in particular a position and orientation (i.e. a pose), of the end-effector tip, in particular of the instrument tip, and to generate an overlapping with the 3D image data on the one hand and the positionally correct overlapped position, in particular pose-correct overlapped position and orientation, of the end-effector tip on the other hand and to output it visually via a display unit and/or to control the end effector on the basis of the overlapping. The tracking system therefore tracks the image unit and identifies a transformation between the KOS of the tracking system and the image unit on the one hand, and on the other hand the tracking system also identifies a transformation between the KOS (the intervention region) of the patient and the KOS of the tracking system, so that a transformation between the image unit and the patient can be determined. In addition, the surgical robot system is adapted to determine the position, in particular the pose, of at least the end-effector tip, in particular of the instrument tip, and thus to determine an exact transformation between the position or pose of the end-effector tip and the patient. Via the patient, the 3D image data of the virtual world can be correlated with the real world of the patient, so that an overlapping of the 3D image data on the one hand and the positionally correct, in particular pose-correct, integration of at least the end-effector tip on the other hand takes place. On the basis of this overlapping, visual assistance can be provided in the form of a displayed overlapping representation, in which at least the position of the end-effector tip is shown in the 3D image data, or a (partially transparent) overlapping representation or side-by-side display of the real image and a corresponding view of the 3D image data is output, or the end effector can be controlled on the basis of the overlapping (using kinematic models, for example), for example along a predefined trajectory. The term ‘position’ refers to a geometric position in three-dimensional space, which in particular is specified using coordinates of a Cartesian coordinate system. In particular, the position can be specified by the three coordinates X, Y and Z. The term ‘orientation’ in turn indicates an orientation (such as at the position) in space. It can also be said that the orientation indicates an alignment with a direction indication or rotation indication in three-dimensional space. In particular, the orientation can be indicated via three angles. The term ‘pose’ includes both a position and an orientation. In particular, the pose can be specified using six coordinates, three position coordinates X, Y and Z and three angular coordinates for the orientation. The term 3D defines here that the image data is spatial, i.e. three-dimensional. The patient's body or at least a part of the body with spatial extension can be digitally available as image data in a three-dimensional space with, for example, a Cartesian coordinate system (X, Y, Z). In other words, the surgical robot system is preferably coupled with an optical imaging system (image unit) in order to visualize and detect the end effectors, in particular instruments, working on the patient's tissue in the intervention region. This makes it possible to track the position, in particular the pose, of the end effector, in particular of the instrument, and to overlap its position, in particular its pose, on a, in particular preoperative, 3D data set (3D image data) such as a computed tomography (CT) or a magnetic resonance imaging (MRI) in order to visually assist the surgeon accordingly or to execute a preoperative intervention plan. In other words, the control unit may use the end effector localization, in particular instrument localization, to overlay the position, in particular pose, of the end effector, in particular of the instrument, in a preoperative 3D data set such as an MR or CT to provide assistance to the surgeon.

According to a further preferred embodiment, the control unit may be adapted to determine, via the optical image of the end effector tip, the position, in particular the position and orientation (i.e. the pose), of the end effector tip, in particular of the instrument tip, relative to the image unit by machine vision, and to determine, via the tracked position and orientation of the optical image unit, the position, in particular pose, of the end effector tip relative to the 3D image data. In other words, the end effector, in particular the instrument, may preferably be localized in space by using the optical image unit to determine the position, in particular pose, by machine vision, the image device being oriented toward the patient and further having the end effector, in particular the instrument, in the field of vision. The surgical robot system is thereby designed to enable port surgery in areas such as neurosurgery, spinal surgery or ENT surgery with a small access where instruments and visualization device have to be inserted through a small opening. In other words, an aspect of the present disclosure is thus a (miniaturized) surgical robot system comprising one or more robots with robot arms, wherein the at least one robot is fastened to a patient fixation unit (a geometric frame structure) that is rigidly connected to the patient. The robot system is further combined with an optical image system (with image unit) which is tracked with/by a tracking system, in particular navigation system, and generates a view of the tissue of the patient with the end-effector tip, in particular the instrument tip in the field of vision, wherein the control unit is adapted to determine (localize) by machine vision the specific position, in particular the pose, of the end-effector tip, in particular of the instrument tip, particularly preferably of the entire instrument, and is used accordingly by the control unit for navigation.

Preferably, the end effector, in particular the instrument, may have a predefined optical marking pattern/marking element/marker, in particular in the form of mutually spaced rings along an end effector axis and/or in the form of a dot pattern and/or in the form of at least two squares and/or in the form of a QR code, and the control unit can be adapted to determine the position, in particular the pose, of the end effector tip, in particular of the entire end effector, using image processing algorithms on the basis of the optical marking pattern detected by the image unit. In other words, the end effector, in particular the instrument, may be provided with optical markings/marking elements such as rings, dots, squares or QR patterns, and the control unit may be adapted to determine the position, in particular the pose, of the end effector, in particular of the instrument, using image processing algorithms. In this way, an instrument tip can also be localized if it is covered by a tissue. Furthermore, an instrument type may be automatically determined via a QR code, for example, for which dimensions or geometric shapes are stored in a memory unit, which are used to determine the instrument tip. Image processing algorithms may also determine the position, in particular pose, of the end-effector tip even better, for example via triangulation.

Furthermore, a rigid body may preferably be arranged on the at least one end effector as an optical marker, which the tracking system detects in order to determine the position and orientation of the end effector tip, in particular of the end effector. In other words, the following (tracking) of the patient and of the optical image unit, in particular of the microscope head of the surgical microscope, can be performed by an optical navigation system comprising a camera in which rigid bodies are provided to objects to be tracked such as the microscope head. In particular, the optical marker may have four spaced IR spheres that the tracking system can detect. In particular, the tracking system may thus be an optical navigation system with optical markers, wherein the optical markers are arranged on the objects to be tracked, in particular the image unit and/or on the end effector.

According to an embodiment, the control unit may be adapted to determine an average value based on the determined position, in particular pose, of the tip of the end-effector tip by machine vision and based on the position, in particular pose, detected by the tracking system to further increase a precision.

According to a further embodiment, the at least one patient fixation unit may be a head holder used in neurosurgery. In other words, the patient fixation unit (the patient-fixing system) may preferably be in the form of a head holder used in neurosurgery. The head holder fixes the patient's head rigidly and thus enables precise intervention.

Preferably, the patient fixation unit may be adapted to be rigidly and immediately connected or fastened to a head of a patient, or to a hip of a patient, or to a knee of a patient, or to an ankle of a patient in order to rigidly fix at least that body portion of the patient to the intervention region relative to the patient fixation unit.

In particular, the patient fixation unit may have a rail, in particular a circumferential rail, particularly preferably an annular rail, to which the at least one robot is fastened via its robot base or is movable translationally via its robot base at least in sections along the rail, in particular in a coupled and loss-proof manner. In other words, the patient fixation unit has a ring-shaped base system or a ring base which is fastened to a base frame of the patient fixation unit, wherein the ring-shaped base system surrounds the surgical intervention region and serves as a base for the at least one robot which is fastened thereto via its robot base. The ring base makes it possible to select and adjust an optimal position for the robot with its robot arm, wherein the robot can be moved along the ring base. In particular, the patient fixation unit has a width, in particular a diameter of the ring base, which substantially corresponds to a height of the patient fixation unit. In particular, the base frame is U-shaped with two opposing legs, at the free ends of which the circumferential rail is rigidly fastened as a ring or connected in one piece of material. In particular, the material of the patient fixation unit is a sterilizable material, in particular stainless steel used in medical technology. In particular, the rail surrounds the intervention region and is arranged above the patient and above an operating table. In particular, a horizontal width of the patient fixation unit is at most 1 meter, preferably at most 50 cm, and a vertical height is at most 1 meter, preferably 50 cm.

According to a further embodiment, the robot base may have a slide with a clamping and/or latching element which is adapted to be guided translationally along the rail and, when the clamping and/or latching element is activated, to fix the position of the robot base relative to the rail in order to adjust various positions of the robot relative to the patient fixation unit. In particular, the slide is guided on the rail so that it cannot be lost and kind of grips around the rail. The clamping and/or latching element may in particular be a manually actuated button which is pre-stressed into a clamping and/or latching state and only transfers the slide into a freely movable state when it is manually actuated against the pre-stressing. In particular, the position of the robot base on the rail, in particular on the circumferential rail, may be adjustable in order to set an optimum position of the robot.

Preferably, the robot arm may have at least five degrees of freedom to align the end effector with a surgical entry path, and/or the end effector itself (at a distal end of the robot arm) may have at least one further degree of freedom, in particular a further hinge with two degrees of freedom (2DOF), in order to allow further movement/articulation within the body of the patient. In other words, the robot arm may have (respectively) at least five or more degrees of freedom to align the end effector with the surgical entry path. In particular, the robot arm has five to seven degrees of freedom. In particular, the end effector at the distal end of the robot arm may thus be provided with further degrees of freedom in order to enable articulation within the patient's body, in particular having a hinge with two degrees of freedom (2-DOF hinge) at the wrist of the robot arm.

Furthermore, in an embodiment, the at least one optical image device may be a digital surgical microscope that provides the optical images of the control unit in a computer-readable manner and further preferably may be a robot-controlled surgical microscope. In other words, the optical image unit is in particular a surgical microscope, particularly preferably a digital microscope, which is in particular moved by a robot-guided microscope arm.

According to a further embodiment, the at least one optical image device may also be an endoscope with a distal image head in order to create an intracorporeal optical image of the patient. Alternatively or additionally, an endoscope may also be used as the at least one optical image unit, in which an image head is located inside the body and can be introduced through the same working channel (port) as the instruments. If, for example, optical marking patterns are provided on the endoscope, a position, in particular pose, of the distal image head of the endoscope as an end-effector tip may also be identified by machine vision using an image through a surgical microscope, as described above.

In particular, the surgical robot system may comprise a surgical navigation system for navigation of the end effector. Preferably, the navigation system may comprise a machine vision-based navigation system. In particular, the navigation system may use a surgical microscope as a camera in order to recognize an end effector, in particular instrument, via image analysis and to determine a position or pose of at least the end effector tip, in particular of the instrument tip. A tracker is no longer necessary for image analysis.

Preferably, the surgical robot system may be adapted to be used for surgery through a port inserted into the patient's body for use in neurosurgery, spinal surgery or ENT interventions.

According to an embodiment, the control unit may be adapted to integrate the specific position, in particular the pose, of the end-effector tip, in particular of the instrument tip, into the 3D image data in the overlapping representation and to output it via a display unit.

Further preferably, the surgical robot system may further comprise a control console (attached to the robot) which is adapted to transmit a manual input by the user/surgeon to the at least one robot-guided end effector and to control the end effector accordingly from a distance. The at least one guided end effector, in particular the instrument, may thus be remotely controlled via a console that translates the surgeon's hand movements or may be maneuvered autonomously on the basis of a preoperative plan. Preferably, the surgical robot system thus comprises a console unit/an input device/a control panel with input devices, which is used by the surgeon in order to transfer the manual movements of the hands to the robot-guided instruments. For example, a joystick may be provided for each hand in order to operate the respective end effector accordingly.

Further preferably, a preoperative intervention plan may be stored in the data provision unit of the surgical robot system, and the control unit may be adapted to control/move the at least one end effector semi-autonomously or completely autonomously on the basis of the overlapping and on the basis of the preoperative intervention plan in order to perform the intervention. In particular, the surgical robot system, in which a preoperative plan, in particular with stored, predefined kinematic models for the individual steps of the intervention to be performed, can thus enable the execution of a preoperative plan and the movement of the instruments by the robot semi-autonomously or fully autonomously.

In particular, the surgical robot system may comprise a computer system with a display for displaying the image of the optical image unit and the position of the instrument overlapped on a CT or MR.

The object of the present disclosure is solved with respect to the control method in that it comprises the steps:
creating, via an optical image device, an optical image of an intervention region of the patient together with an end-effector tip of a robot-guided end effector, in which the robot is directly connected to a patient fixation unit, and providing the optical image;
providing, via a data provision unit, digital 3D image data of the patient;
tracking, via a tracking system, of at least the optical image unit and a body portion of the patient fixed relative to the patient fixation unit; and
determining, via a control unit, a position, in particular a position and orientation, of the end-effector tip, in particular the instrument tip, relative to the image unit, in particular by machine vision;
creating an overlapping with the 3D image data on the one hand and the pose-correct position, in particular position and orientation, of the end-effector tip on the other hand; and
outputting the overlapping as an overlapping representation by a display unit and/or controlling the end effector on the basis of the overlapping. By the control method according to the present disclosure a position may be outputted, accordingly.

With respect to a computer-readable storage medium, the objects are solved by comprising instructions which, when executed by a computer, cause the computer to perform the method steps of the control method according to the present disclosure.

Any disclosure related to the surgical robot system according to the present disclosure also applies to the control method of the present disclosure and vice versa. In particular, the control unit of the robot system may be adapted to perform the steps of the control method.

### BRIEF DESCRIPTION OF THE DRAWINGS

The present disclosure is explained in more detail below with reference to preferred embodiments with the aid of Figures.

FIG. 1 shows a perspective view of a surgical robot system according to a first preferred embodiment;

FIG. 2 shows a perspective view of a surgical robot system according to a further, second preferred embodiment;

FIG. 3 shows a detailed schematic perspective view from FIG. 2 with the robot-guided instrument, which has rings as a marking pattern that is detected via the microscope head in order to determine the position of the instrument tip; and

FIG. 4 shows a flow diagram of a control method according to a preferred embodiment.

### DETAILED DESCRIPTION

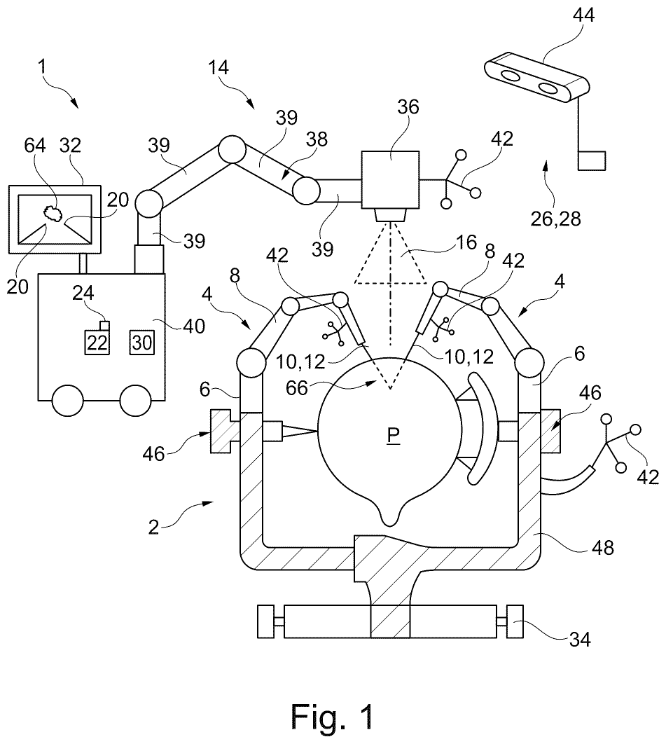

FIG. 1 shows a schematic perspective view of a surgical robot system 1 according to a first preferred embodiment of the present disclosure. The surgical robot system 1 is used in the present case in a craniotomy for a neurosurgical intervention on a head of a patient P in order to manipulate the brain tissue of the patient P with particular precision.

For the intervention, the patient's head P, as the body part or body portion to be fixed, on which the surgical intervention takes place, is rigidly connected to a patient fixation unit 2. In this context, rigid means that the patient's cranium is fixed in a pose/fixed position in relation to the patient fixation unit 2 and does not change. In this way, the intervention region on the patient is fixed statically in relation to the patient fixation unit 2 so that the geometric relative relations are maintained even if the patient P is moved on an operating table.

In this embodiment, the surgical robot system 1 has two miniaturized surgical robots 4 that can be actively controlled. These robots 4 are configured/designed in the same way and each have a robot base 6, which are directly and immediately fixed/fastened to the patient fixation unit 2 on two opposite sides as seen opposite the head of the patient P and represent the local attachment point of the robot 4 to the patient fixation unit 2.

A movable, multi-segmented robot arm 8, in this case with four robot arm members, is articulated to the robot base 6 with a terminal/front end effector 10 in the form of a surgical instrument 12. The robot arm 8 can be actively controlled and can guide the instrument 12 accordingly. Since the two robots 4 are fixed directly to the patient-fixing system 2, the distance between the head of the patient P (with the intervention region) and the surgical instrument 12 is minimized, which particularly improves the precision of actuation, and the two robots 4 can also be designed due to the immediate proximity to the intervention region in such a way that the robot arms 8 only require a short reach, with associated smaller lever arms (thus lower lever effects) and can thus be built more compact and smaller. It is also possible to integrate several, in this case two, small robots 4 into the limited volume, which also has to provide sufficient space for the surgeon to view or even manipulate manually. This improves the intervention, since an actuation arm of the robot 4 is now available for each of the surgeon's hands and the surgeon can perform dual simultaneous manipulation.

The surgical robot system 1 also has an optical image unit 14 in the form of a robot-guided surgical microscope, which is configured to create and digitally provide an optical image A of the intervention region of the patient P together with an intracorporeal tissue via an optical system and an image sensor. In addition, the surgical instrument 12, or at least an instrument tip 20 as end-effector tip 18, which performs the manipulation of the tissue, is located in the field of vision 16 of the image unit 14. The optical image unit 14 captures this instrument tip 20, which is guided by the surgeon in his field of vision 16, in the created image A accordingly.

In order to navigate via a port and with only a small visible opening during the intervention and to correlate the detected instrument tip 20 with preoperative 3D images 3DA, the surgical robot system 1 furthermore has a data provision unit 22 with a memory unit 24, in which digital preoperative 3D image data 3DA of the patient P with at least the body portion on which the intervention is to take place is stored in the form of a magnetic resonance imaging (MRI) or a computer tomography (CT). This data provision unit makes this 3D image data 3DA available in digital or computer-readable form. In order to carry out a correlation between the real world with the patient P and the virtual world with the 3D image data of the patient P, a tracking system 26 is also required, which in this embodiment is designed as a surgical navigation system with optical three-dimensional recognition. The tracking system 26 is adapted to spatially detect the optical image unit 14, in particular a head of the optical image unit, and to track it in space. The tracking system 26 is further adapted to spatially detect the fixed head of the patient P, indirectly via the detection of the patient-fixing system 2 with predefined contact points to the head of the patient P and corresponding forward determination. A relation of the patient P's head to the patient fixation unit 2 may also be predefined.

Since the tracking system 26 spatially detects both the instrument 12 and the head of the patient P with the intervention region, a specially adapted control unit 30 can correlate the preoperative 3D image data 3DA with the head of the patient P and thus with the patient P, so that an overlapping of the real world with the virtual world, in particular the real images with the virtual preoperative images, can be performed and is provided to the surgeon for navigation during the intervention via the surgical robot system 1.

In this embodiment, the surgical robot system 1 is specially configured to determine the position and orientation, i.e. the pose, of the end-effector tips 18, here the instrument tips 20, and to generate these identified instrument tips in an overlapping or overlapping representation with the 3D image data 3DA of the intervention region on the one hand and a superimposed pose-correct overlapping on the other hand and to output them visually together by a display unit 32 in the form of an operating theater monitor. This provides the surgeon with particularly advantageous navigation with the capability of appropriate control. Since the overlapping representation can be duplicated as desired, the surgeon can, for example, put on 3D glasses in which the overlapping representation is displayed in three dimensions and, sitting at an external console with input devices far away from the intervention region, control the robots 4 on the basis of his visual display. The overlapping can also be used by the control unit 30 to control the instrument semi-autonomously or autonomously according to an intervention plan. The present surgical robot system thus creates the possibility of telemedicine in particular.

In this embodiment, the patient fixation unit 2 is firmly connected to an operating table 34 in order to ensure the necessary stability.

The optical image unit 14 in the form of the surgical microscope has a terminal microscope head 36, which comprises the optical system and the image sensor, and which can be actively adjusted via a multi-membered, robot-guided microscope arm 38 with several microscope arm segments 39 in order to maneuver into predetermined poses during the operation and to have the best possible view. In particular, the surgical microscope can move around a focal point in this way in order to provide different views. The microscope arm 38 is articulated to a mobile rolling cart 40, which can be positioned at different locations in an operating room depending on the requirements of an intervention.

In order to spatially detect and track the microscope head 36, a passive optical marker 42 in the form of a rigid body with several (in particular four) IR marking spheres spaced apart from each other is arranged on its housing. A stereo camera 44 of the tracking system 26 detects this marker 42 and can use it to detect a pose in space relative to the stereo camera 44. In addition, an optical marker 42 with IR marking spheres is fastened to the last segment of the robot arm 2, i.e. immediately adjacent to the instrument 12, so that the stereo camera can also determine the pose of the last segment of the robot arm 2 and, due to the predefined relation to the instrument 12, also the pose of the instrument 12 and an instrument tip 20. Finally, a corresponding marker 42 is also provided on the patient fixation unit 2. The position and orientation of the instrument tip 20 is then correlated with the 3D image data 3DA by the control unit 30.

In this embodiment, the data provision unit 22, the memory unit 24 and the control unit 30 are integrated in the rolling cart, but it is of course also possible to connect the surgical robot system 1 to a specially adapted computing unit via a local network connection, for example, which can be provided both decentrally and centrally.

The patient-fixing system 2 has a U-shaped frame 48 for the rigid fixation of the head of the patient P with two opposite legs, on each of which adjustable fixing pins 46 are provided facing each other, which can be screwed in toward each other in order to reduce the distance between the fixing pins 46 and to clamp the head of the patient P between them accordingly. On the left side in FIG. 1, a single protruding bolt-like fixing pin 46 with a tip on the front side is provided, while on the opposite side a circular section-shaped arc with at least two fixing pins 46 aligned radially inward is provided, wherein the arc can be moved and adjusted translationally inward toward the head and outward away from the head again via a screw thread. In this way, the patient fixation unit 2 with the three fixing pins 46 can be adapted to different head sizes. The three fixing pins 46 allow the pose of the patient's head P in relation to the fixation unit 2 to be determined without under-determination or over-determination.

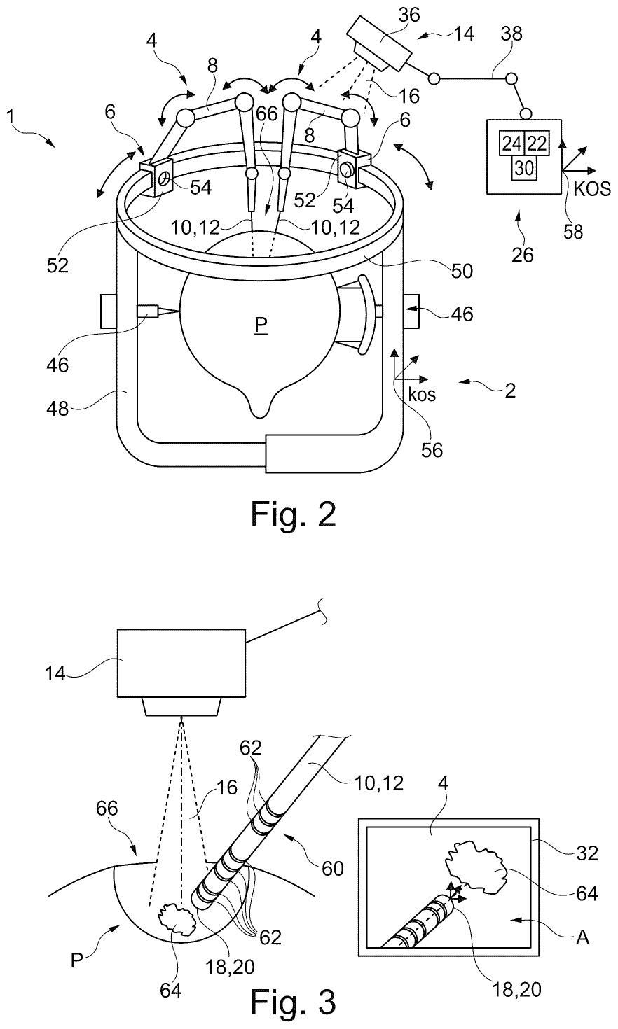

FIG. 2 shows a perspective view of a surgical robot system 1 according to a further, second embodiment and FIG. 3 shows a detailed partial view of the corresponding intervention region. This embodiment differs from the first embodiment in the configuration for determining the position and orientation of the instrument tip 20 as well as in the connection of the robots 4 to the patient fixation unit 2.

In this embodiment, the patient fixation unit 2 has a circumferential, circular rail 50 on its U-shaped frame 48 on an upper side as seen in FIG. 2, wherein the circular rail 50 is rigidly connected to the frame 48 and rigidly fixed relative to the head of the patient P. Matching the rail 50, the robot base 6 in this embodiment is designed as a slide 52, which geometrically embraces the rail 50 and can be guided along the longitudinal axis of the rail 50. In this way, the position of the robot base 6 in relation to the patient fixation unit 2 can be changed and easily adapted for an intervention. Furthermore, the robot base 6 also has a clamping and latching element 54, which is adapted to clamp the slide 54 resiliently relative to the rail 50 on the one hand and to additionally fix it via a latching mechanism on the other hand. In particular, the clamping and latching element 54 may be designed in the form of an operating button, so that when this operating button is pressed, the slide 54 can slide along the rail 50 and when the operating button is released, it is resiliently pressed back into its initial position, in which it clamps the slide 54 to the rail 50 and latches it to the rail 50. In particular, the robot base 6 may be designed to be coupleable and decoupleable, so that it can be coupled to the rail 50 and can be decoupled again if necessary. In this way, the number of robots 4 connected to the patient fixation unit 2 can be adapted to the intervention as required. In this embodiment, the surgical robot system 1 has two robots 4, as in the first embodiment. In an embodiment that is not shown, a motor is also installed in the robot base in order to actively move the slide 52 on the circular rail.

In contrast to the first embodiment, however, no optical markers 42 are provided on the terminal segments of the robot arms 8; instead, the position of the instrument tip 20 is identified by machine vision, as explained below. On the one hand, the KOS of the patient P of the control unit 30 is known and, on the other hand, the KOS of the rolling cart 40 of the surgical microscope is known. Using kinematic relations, the control unit 30 can determine the KOS of the microscope head 36 with the image sensor based on the KOS of the rolling cart 40.

The microscope head 36 itself optically detects the instrument tip 20 of the instrument 12 via the optical image A, and the control unit 30 is adapted to determine a position and an orientation of this instrument tip 20 via machine vision, even if only a 2D image is provided. This has the advantage that an instrument tip can be determined particularly precisely and can be localized in relation to the 3D image data 3DA. Furthermore, this configuration has the advantage that no optical markers need to be provided in the already cramped intervention region.

FIG. 3 shows a schematic, detailed partial view from FIG. 2 with the microscope head 36 on the one hand and the instrument 12 on the other hand. For an even better determination of the position and orientation, the cylindrical surgical instrument 12 has concentric optical rings 62 spaced along its longitudinal axis, which are arranged in a special pattern to each other and represent an optical marking pattern/marking element 60 for the determination. Based on the predefined optical pattern, the control unit 30 can determine the position and orientation in the image A even more precisely. If the instrument tip 20 is immersed in a tissue of the patient, covered by the tissue and thus not optically visible, the control unit 30 can still infer the tip of the instrument 12 on the basis of the optical marking elements 60. In addition, the robot-guided microscope head 36 may also be adapted to perform even a small movement in order to create different views of the instrument 12 in order to determine the position and orientation even better via machine vision.

As shown in FIGS. 1 to 3 above, small robots 4 with robot arms 8 are fastened directly to a head holder system or a patient fixation unit 2, which ensures a rigid connection between the robot base 6 and the patient P. The robot arms 8 have five to seven degrees of freedom in order to align the surgical instruments 12 within the surgical access, in particular via a port in neurosurgery. In addition, the surgical instruments 12 themselves may have further degrees of freedom, for example to realize a curved tip that can move within the patient's body P. The surgical robot system 1 is combined with or has an optical imaging system such as a surgical microscope. A tracking system 26, in particular navigation system 28, is then used to track the patient P and the surgical microscope, for example via rigid bodies as optical markers, which are fastened to the patient fixation unit 2 (on the head holder) on the one hand and to the housing of the microscope head 36 on the other hand. The surgical instrument 12 is also tracked, either directly by attaching a further rigid body as an optical marker to the robot end effector or by performing a machine vision procedure. In the case of machine vision based on image processing, no rigid bodies are required on the robot or instrument 12. Instead, the instrument tip 20, which is visible in the field of vision of the optical imaging system 14, is provided with an optical pattern, in particular with rings, dots, squares or QR patterns—each of which makes it possible to identify the type and position of the instrument 12 relative to the optical imaging system 14. Since the optical imaging system 14 and the patient P are localized by the navigation system 28, the position, in particular the position and orientation of the instrument 12 can be displayed on a preoperative 3D data set or preoperative 3D image data 3DA of the patient, such as a CT image or an MR image.

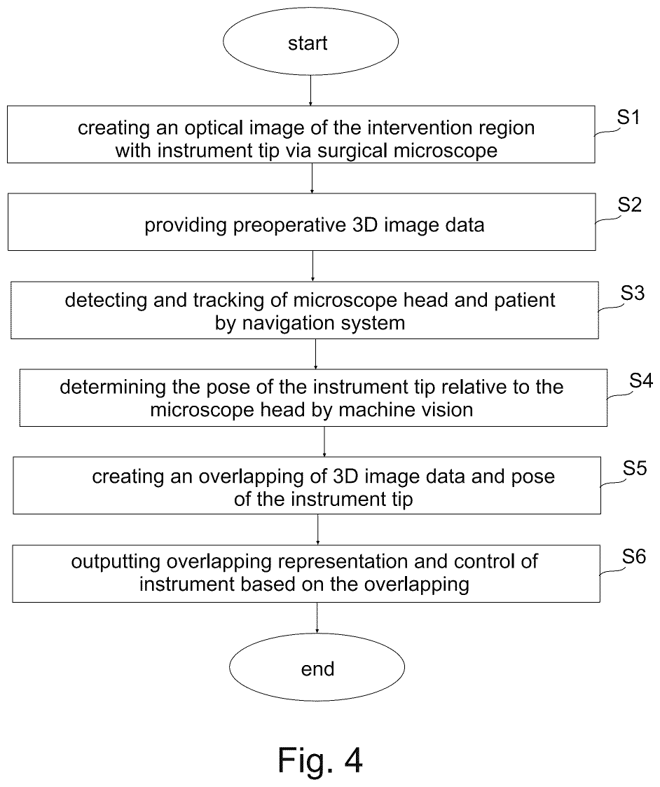

FIG. 4 shows a flowchart of a control method according to a preferred embodiment. This control method can also be stored in a computer-readable storage medium in the form of instructions, which can be used in the surgical robot system 1 of the first or second embodiment.

In a first step S1, the control method creates an optical image A of an intervention region of the patient P together with an end-effector tip 18 of a robot-guided end effector 10 via an optical image device 14. Here, the robot 4 is directly connected to a patient fixation unit 2. The optical image A is provided in computer-readable form according to a control unit 30.

In a step S2, digital 3D image data 3DA of the patient P is also provided to the control unit 30. This means that both real and virtual digital images of patient P are available.

In a subsequent step S3, the optical image unit 14 is then tracked directly by a tracking system 26, and a body portion of the patient P, which is fixed relative to the patient fixation unit 2, is determined once or continuously relative to the tracking system in space via the patient fixation unit with a predetermined relationship to the body portion, in particular to a head of the patient, so that a correlation of the virtual world (3D image data) and the real world (optical image; patient and other tracked objects) can be carried out.

Then, in a step S4, the control unit 30 determines a position and orientation of the instrument tip (20) relative to the image unit (14) by machine vision.

In a step S5, a correlation or overlapping with both the 3D image data on the one hand and the pose-correct position and orientation of the instrument tip 20 on the other hand is identified or created.

Finally, in a step S5, the overlapping U is output as an overlapping representation by the surgical monitor in order to visually support a user and, in addition, semi-autonomous control of the instrument 10 is performed on the basis of the overlapping U. In this embodiment, an intervention plan has been determined prior to surgery and individual successive steps of this intervention plan have been defined in advance. The surgeon can confirm a first step of the control via a control console, which is at a distance from the robot, so that the instrument 12 independently executes this first step. After completion of the first step, the surgeon can check the correct execution and, if necessary, make a local correction manually with a hand instrument, for example, and proceed to the next step of controlling the instrument 12 via a confirmation input. In this way, similar to a discrete, staged procedure, the intervention can finally be carried out. The position and orientation of the instrument tip is continuously determined and used for appropriate control.

### LIST OF REFERENCE SIGNS

1 surgical robot system
2 patient fixation unit
4 surgical robot
6 robot base
8 robot arm
10 end effector
12 surgical instrument
14 image unit
16 field of vision of image unit
18 end-effector tip
20 instrument tip
22 data provision unit
24 memory unit
26 tracking system
28 navigation system
30 control unit
32 display unit
34 operating table
36 microscope head
38 microscope arm
39 arm segment
40 rolling cart
42 optical marker
44 stereo camera
46 fixing pins
48 fixing frame
50 rail
52 slide
54 clamping and fixing element
56 KOS patient
58 KOS microscope
60 marking pattern
62 ring
64 tissue
3DA 3D image data
A optical image
P patient
S1 step of creating optical image
S2 step of providing 3D image data
S3 step of tracking the image unit and the patient
S4 step of determining pose of instrument tip by machine vision
S5 step of creating overlapping of 3D image data and pose of instrument tip
S6 step of outputting overlapping representation and control of instrument based on overlapping

1. A surgical robot system for surgical intervention on a patient, comprising:
at least one patient fixation unit which is adapted to be rigidly and directly attached to the to a head of the patient, in order to rigidly fix at least a body portion of the patient with an intervention region relative to the at least one patient fixation unit;
at least one surgical robot that is controllable, the at least one surgical robot comprising a robot base, which is directly connected to the at least one patient fixation unit, as well as a robot arm that is moveable and connected to the robot base and an end effector; and
a surgical navigation system for navigating the end effector.

2. The surgical robot system according to claim 1, wherein the surgical robot system further comprises:
at least one optical image unit, which is adapted to create an image of the intervention region and an end effector tip, and to provide the image in a computer-readable manner;
a data provision unit, which is adapted to provide digital 3D image data of the patient;
a tracking system, which is provided and adapted to detect and track in space at least the at least one optical image unit and at least the body portion of the patient, which is fixed relative to the at least one patient fixation unit, and
a control unit, which is provided and adapted to determine a position of the end effector tip and to generate an overlapping with the digital 3D image data and a positionally correct overlapped position; of the end effector tip and to output the overlapping visually via a display unit as an overlapping representation and/or to control the end effector based on the overlapping.

3. The surgical robot system according to claim 2, wherein the control unit is adapted to determine, via an optical image of the end effector tip, the position, of the end effector tip, relative to the at least one optical image unit by machine vision, and to determine, via a pose of the at least one optical image unit tracked by the tracking system, the position, of the end effector tip relative to the digital 3D image data.

4. The surgical robot system according to claim 2, wherein the end effector has a predefined optical marking pattern, and the control unit is adapted to determine the position of the end effector tip based on the predefined optical marking pattern detected by the at least one optical image unit.

5. The surgical robot system according to claim 2, wherein an optical marker is arranged on the at least one end effector or on a terminal member of the robot arm, wherein the optical marker is detected and tracked by the tracking system in order to determine the position and orientation of the end effector tip.

6. The surgical robot system according to claim 1, wherein the at least one patient fixation unit is a head holder used in neurosurgery.

7. The surgical robot system according to claim 1, wherein the at least one patient fixation unit has a circumferential rail, to which the at least one surgical robot is fastened via the robot base or is moveable translationally along the circumferential rail via the robot base at least in sections.

8. The surgical robot system according to claim 7, wherein the robot base has a slide with a clamping and/or latching element which is adapted to be guided translationally along the circumferential rail and, when the clamping and/or latching element is activated, to fix a position of the robot base relative to the circumferential rail and, when the clamping and/or latching element is deactivated, to be freely movable again in order to adjust various positions of the at least one surgical robot relative to the at least one patient fixation unit and thus relative to the patient.

9. The surgical robot system according to claim 1, wherein the robot arm is configured to have at least five degrees of freedom to align the end effector with a surgical entry path, and/or the end effector itself has at least one further degree of freedom, in order to allow further articulation within the patient.

10. The surgical robot system according to claim 2, wherein the at least one optical image unit is a digital surgical microscope that provides an optical image of the control unit in a computer-readable manner.

11. The surgical robot system according to claim 2, wherein the at least one optical image unit is an endoscope with a distal image head, which is adapted to create an intracorporeal optical image of the patient.

12. The surgical robot system according to claim 1, wherein the surgical robot system further comprises an external control console which is separate from the at least one patient fixation unit with the at least one surgical robot, and which comprises manual gripping control means for transmitting a manual input by a user to the end effector and controlling the end effector accordingly from a distance.

13. The surgical robot system according to claim 2, wherein a preoperative intervention plan is furthermore stored in the data provision unit of the surgical robot system, and the control unit is adapted to control the end effector semi-autonomously or completely autonomously based on the preoperative intervention plan in order to perform the surgical intervention.

14. A control method for a surgical robot system that includes a robot, the control method comprising the steps of:
creating, via an optical image device, an optical image of an intervention region of a patient together with an end-effector tip of a robot-guided end effector, wherein the robot is directly connected to a patient fixation unit rigidly connected to the patient, and providing the optical image;
providing, via a data provision unit, digital 3D image data of the patient;
tracking, via a tracking system, of at least the optical image device and a body portion of the patient fixed relative to the patient fixation unit;
determining, via a control unit, a position of the end-effector tip relative to the optical image device by machine vision based on the basis of the optical image;
creating, via the control unit, an overlapping with the digital 3D image data and the position of the end-effector tip; and
outputting the overlapping as an overlapping representation by a display unit and/or controlling the robot-guided end effector based on the overlapping.

15. A computer-readable storage medium comprising instructions which, when executed by a computer, cause the computer to perform the control method according to claim 14.
